(19) United States
(12) Patent Application Publication
(10) Pub. No.: US 2024/0310365 A1
(43) Pub. Date: Sep. 19, 2024

(54) DEVICE FOR EVALUATING INNER EAR DRUG DELIVERY

(71) Applicant: North Carolina State University, Raleigh, NC (US)

(72) Inventors: Alon Grinbaum, Raleigh, NC (US); Adele Moatti, Raleigh, NC (US); Frances Ligler, Raleigh, NC (US)

(21) Appl. No.: 18/603,792

(22) Filed: Mar. 13, 2024

Related U.S. Application Data
(60) Provisional application No. 63/489,905, filed on Mar. 13, 2023.

(51) Int. Cl. G01N 33/50; C12M 1/00; C12M 1/12; C12M 1/34; C12M 3/00

(52) U.S. Cl. CPC G01N 33/5082; C12M 21/08; C12M 23/34; C12M 25/02; C12M 41/32; G01N 2500/10

(57) ABSTRACT

The present disclosure relates generally to test devices comprising a round window membrane and/or an oval window membrane separating two compartments and to the use of such devices in methods for identifying compounds and compositions suitable for use as therapeutics via intratympanic administration.

### CROSS-REFERENCE TO RELATED APPLICATIONS

This application claims the benefit of priority of U.S. Provisional Patent Application No. 63/489,905, filed Mar. 13, 2023, the disclosure of which is incorporated herein by reference in its entirety.

### STATEMENT REGARDING FEDERALLY SPONSORED RESEARCH

This invention was made with government support under DC020005 and DC019960 awarded by the National Institutes of Health. The government has certain rights in the invention.

### BACKGROUND OF THE DISCLOSURE

### Field of the Invention

This disclosure relates to methods and devices for evaluating delivery of therapeutic compounds and compositions to the inner ear.

### Description of Related Art

Hearing loss affects nearly 20% of the global population. Several studies link hearing loss to the increased rate of dementia, Parkinson's disease, depression, and more. To mitigate the detrimental effects of hearing loss, hearing aids, and cochlear implants are commonly used, with limited success. Cochlear implants have their limitations. Not only can cochlear implant users lose their residual hearing and face other surgical problems, but some fail to hear in noisy environments, or lose the capacity to enjoy music. Hearing aid users also suffer from a lack of normal auditory perception. Consequently, researchers are dedicated to developing treatments to restore hearing or prevent hearing loss.

A major obstacle to developing novel treatments, e.g., intratympanic treatments, for diseases and disorders of the ear is the lack of effective and minimally invasive procedures to deliver substances to the inner ear. Systemic delivery is challenging as only a fraction of the therapeutic passes through the blood labyrinth barrier and reaches the inner ear. Delivery via intracochlear and intratympanic procedures seems promising, but these procedures have major shortcomings. Intracochlear methods inject therapeutics directly into the cochlea during surgery, and they increase the risk of hearing loss through cerebrospinal and perilymph fluid leakage. Additionally, using intracochlear delivery methods results in large variability in the therapeutic outcomes since such surgeries are technically difficult and individual results are variable. Also, some intracochlear approaches suggested for inner ear therapy in small animals—e.g., through the semicircular canal are not practical in humans. The intratympanic injection method is less invasive and causes minimal damage to the inner ear; intratympanic injection is used regularly to deliver steroids such as dexamethasone phosphate and gentamicin to treat idiopathic sudden sensorineural hearing loss and Menière's disease. However, intratympanic injection is less effective in delivering substances with large molecular weight, negative change, or hydrophilic properties to the inner ear, since the injected substances that fill the inner ear cavity must pass through a round window membrane (RWM) and/or an oval window membrane (OWM).

Thus, there is a need for the administration to the car via simple delivery of therapeutic compositions through the round window membrane and/or oval window membrane where the therapeutic passes through the round window and/or oval window membrane. Importantly, the need addressed herein does not include passage of therapeutic compositions through the tympanic membrane.

The RWM is an epithelial barrier that consists of three layers. The first and outer layer, facing the middle ear, consists of epithelial cuboidal cells with apical, laterally placed tight junctions. The tight junctions prevent the passage of most molecules. The second layer is made of fibroblasts, collagen, and elastic fibers which contain blood and lymph vessels as well as nerve endings. The third layer consists of wide and flat inner epithelial cells, facing the inner car (as shown in FIG. 1a). The RWM is only permeable to specific molecules, and while some substances and compositions can pass through the RWM, most therapeutics have low or no penetrance. The RWM is considered the major barrier that limits drugs from reaching the inner car.

The oval window (OW) is a membrane or ligament covered opening from the middle car (the tympanic cavity) into the inner ear. The OWM offers an additional route for drug passage to the inner ear.

Therefore, there is a need for an ex-vivo RWM and/or OWM model to readily identify therapeutic compositions that may be directly delivered to the inner car following intratympanic administration. The manipulation of delivery techniques to improve the permeability can also be tested using the disclosed device.

### SUMMARY OF THE DISCLOSURE

The following embodiments and aspects thereof are described and illustrated in conjunction with compositions and methods which are meant to be exemplary and illustrative, not limiting in scope.

In one aspect, the present disclosure provides a test device comprising a first compartment and a second compartment, wherein the first compartment is fluidly connected to the second compartment by an aperture between the first compartment and the second compartment; and a mammalian round window membrane (mRWM), a mammalian oval window membrane (mOWM), or a combination of a mRWM and a mOWM, and no tympanic membrane; wherein the membrane is provided over the aperture such that the membrane isolates the first compartment from the second compartment. The membranes of the device are only RWMs, OWMs or combinations thereof. No tympanic membrane is present in the device. Thus, the device simulates administration of a compound or composition by intratympanic injection through the tympanic membrane and into the inner ear. The device therefore can be used to quantitatively and qualitatively determine the permeability of compounds and compositions through RWM and/or OWM.

Also provided is a test device as described above, and wherein the first compartment further comprises a first solution; and the second compartment further comprises a second solution, optionally of a volume different than that of the first solution, wherein the solution in the second compartment is the same or different than the solution in the first compartment.

Also provided is a method of identifying a compound or composition comprising a compound suitable for use as a therapeutic via intratympanic administration to treat an car disorder or disease, or a genetic disorder in a mammalian subject, the method comprising applying a test compound to a test device as disclosed herein and measuring the amount of test compound that passes through the membrane into the second compartment. The test compound can be applied to the device alone or as a component of a composition.

Also provided is a method of treating an inner ear disease or disorder in a subject, the method comprising administering to a subject in need thereof a therapeutically effective amount of a compound identified by the method of identifying a compound suitable for use as a therapeutic via intratympanic administration to treat an car disorder or disease, or a genetic disorder in a mammalian subject, the method comprising applying a test compound to the test device. These methods identify compounds or compositions suitable for use as therapeutics via intratympanic administration of a therapeutic alone or as a component of a liquid composition to treat an car disorder or disease in at least the inner ear. Intratympanic administration as used herein is administration into the middle car, typically by injection but can also include car drops. Thus, intratympanic administration as that term is used herein involves injection of a therapeutic compound or therapeutic composition through the tympanic membrane (ear drum) into the tympanic cavity. Intratympanic administration also encompasses delivery of a therapeutic compound or therapeutic composition through a tympanostomy tube (an “ear tube”) into the middle car. Once in the middle car, the composition then passes through the mRWM and/or mOWM into the inner ear and exerts its therapeutic effect.

Also provided is a method of assembling the test device described above, the method comprising securing a membrane, either a mRWM, mOWM or a combination thereof, specifically excluding the tympanic membrane, over an opening of a first compartment, wherein the first compartment is fluidly connected to a second compartment.

In addition to the exemplary aspects and embodiments described above, further aspects and embodiments will become apparent by reference to the drawings and by study of the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

The accompanying drawings are included to provide a further understanding of the methods and compositions of the disclosure, and are incorporated in and constitute a part of this specification. The drawings illustrate one or more non-limiting embodiment(s) of the disclosure, and together with the description serve to explain the principles and operation of the disclosure.

FIG. 1a-i. shows the microstructure and cellular constitution of a porcine Round Window Membrane (pRWM). FIG. 1a shows a TEM micrograph of a pRWM which corresponds to the schematic in FIG. 1b. FIG. 1b shows a schematic of the disclosed porcine RWM that shows a three-layer structure consisting of the outer epithelial (OE) layer, fibroblast (F) layer, and inner epithelial (IE) layer. FIG. 1c shows a TEM micrograph of the porcine OE cells that form tight junctions that restrict the passage of molecules. FIGS. 1d-1f show the TEM Microvilli (MV) and cilia (Ci) structures on the edges of the OE cells. FIG. 1f shows the cilia in the canonical 9+2 structure. FIG. 1g shows TEM micrograph of the fibroblast cells and the CF in their vicinity. FIG. 1h shows the TEM micrograph of the IE layer which includes squamous epithelial cells. FIG. 1i shows immunostaining of the porcine RWM using epithelial (anti-EpCAM) and fibroblast (anti-Vimentin) cell markers.

FIG. 2a shows a schematic of the inner ear, the dashed circle marks the RWM location, which can be excised using a dental drill. FIG. 2b shows a schematic of the excised RWM with the three-layer structure and the surrounding bone. FIG. 2c is a side cross-sectional view of a RWM device as described herein. Substances to be tested can be included in the fluid in the top compartment; substances that pass through the RWM can be sampled from the bottom compartment at various time points. FIG. 2d shows photographs of the real-life postnatal day 0 (P0) porcine inner ear, an excised RWM inserted inside a testing device, and test substances in the upper compartment of the device. FIG. 2e shows the result of the Alamar blue metabolic activity test which is reported as a percent reduction of the Alamar blue (mean±SD), with higher levels of reduction indicating good cell viability in the RWM. FIG. 2f shows dexamethasone (˜400 μg/ml) passage through the RWM over a period of 24 hr. FIG. 2g shows the corresponding calculated permeability (Kₚ) of dexamethasone (mean±SD) based on the measured concentrations in FIG. 2f.

FIG. 3a-c. FIG. 3a is a schematic representation of passage of dexamethasone (Dex) and dexamethasone-fluorescein (DexF) through pRWM excised at different postnatal ages of 0 days (P0), 20 days (P20), and 40 days (P40). FIG. 3b shows the passage of 0.5 mM (˜500 μg/ml) DexF and 1 mM (˜400 μg/ml) Dex through the RWM over a period of 24 hr. (mean±SD). FIG. 3c shows the calculated permeability (Kₚ) of DexF and Dex (mean±SD).

FIG. 4a-c. FIG. 4a is a schematic representation of the differences in the RWM structure between healthy (80-100 percent reduction: PR), leaky but healthy (including microscopic holes or tears), and lower viability (15-30 PR) explants. FIG. 4b shows the passage of 0.5 mM DexF that was tested over a period of 24 hr. on n=3 healthy (80 PR), n=3 leaky, and n=3 low-viability porcine RWM explants (mean±SD). FIG. 4c shows the calculated permeability (Kₚ; mean±SD).

FIG. 5a-d. show several steps in the fabrication of a test device as described herein. FIG. 5a shows cutting of the inner ear to excise the RWM starts from the lateral and then the base of the inner car. FIG. 5b shows removal of the RWM together with a portion of the supporting bone from the dorsal part of the car. FIG. 5c This image shows mounting of the RWM on top of a cut Eppendorf tube. FIG. 5d shows adhering the RWM to the Eppendorf tube with adhesive to cover the entire aperture into the tube.

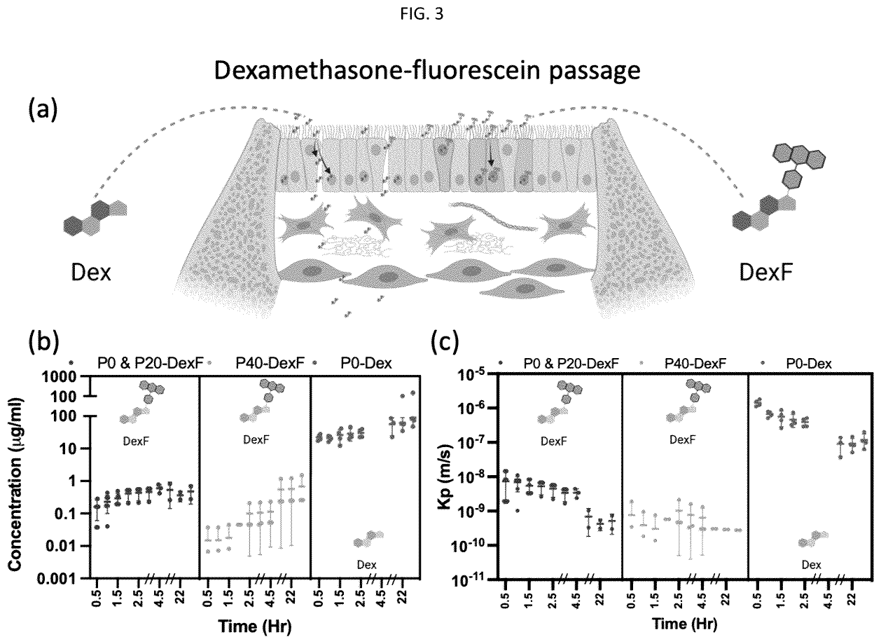

FIG.6a-d. FIG. 6a shows DexF (0.5 mM) passage through the RWM with (blue) and without (red) collagenase I treatment (80 mg/ml; n=3 P0; n=1 P40; mean±SD). RWM was excised at different postnatal ages of 0 days (P0), and 40 days (P40). FIG. 6b shows the corresponding calculated permeability, Kₚ (mean±SD). FIG. 6c shows a DexF (0.5 mM) passage through the RWM without and with saponin treatment at various concentrations 130 μg/ml, 390 μg/ml, 1300 μg/ml. FIG. 6d shows the corresponding calculated permeability, Kₚ (mean±SD).

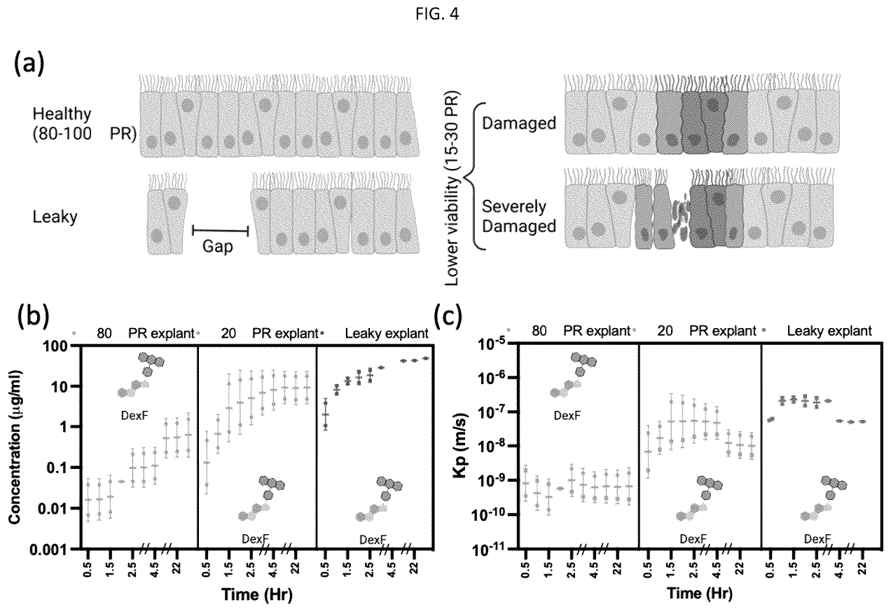

FIG. 7a-b shows a schematic representation of one embodiment of the disclosed invention. FIG. 7a shows the first compartment (101) and the second compartment (102) are separated by the membrane (103) (either a RWM, an OWM or a combination of a RWM and OWM). A test compound is added to the first compartment (101) through an opening (104) and allowed to pass through the membrane (103). The test compound can be collected or otherwise detected and measured in the second compartment (102) wherein compound concentration and behavior through the membrane can be tested. FIG. 7b shows the membrane (103) supported by the inner car bone (201). The inner car bone is secured to the sides (204) of the second compartment by an adhesive (202), although it may alternatively be secured to the first compartment (203).

FIG. 8 is a top view of a test device as described herein having a membrane which is a combination of a RWM and an OWM provided between the first and second compartments.

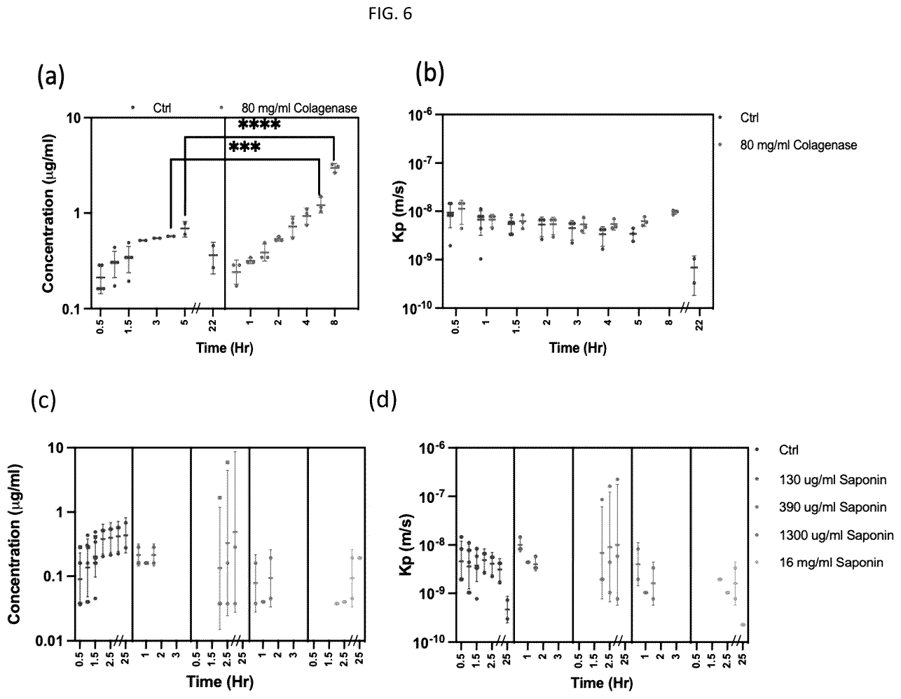

FIG. 9a. is a graph comparing administration of dexamethasone (100 μg/ml) to a RWM in a device of this disclosure (ex vivo) with intratympanic injection of dexamethasone (again 100 μg/ml) to porcine inner ear (in vivo) as described in Example 5.

FIG. 9b. shows a simulation of the dexamethasone (Dex) removal from the middle car after 0.5 hour and the entrance of Dex to the scala tympani over 30 min. The simulation was generated using FluidSim4 software (commercially available from TURNER SCIENTIFIC) based on volumetric data from the porcine inner ear. The simulation of Dex concentration in scala tympani is in line with our in vivo measured concentration.

FIG. 9c. is a graph of in vivo permeability (Kp) determined for dexamethasone, when middle ear fluid removal processes were accounted for according to Example 5, compared with the ex vivo permeability (Kp) for dexamethasone determined herein.

### DETAILED DESCRIPTION OF THE DISCLOSURE

For the purposes of explicating and understanding the principles of this disclosure, reference is made to embodiments and specific language used to describe the same. The skilled artisan will nevertheless understand that no limitation of the scope of the disclosure is thereby intended, such alteration and further modifications of the disclosure as illustrated herein, being contemplated as would normally occur to one skilled in the art to which the disclosure relates.

### Definitions

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the meaning commonly understood by one of ordinary skill in the art.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, clement, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

The term “biocompatible material”, as used herein, refers to any material that is not harmful to living tissue.

The term “biocompatible sealant”, as used herein, refers to any sealing material or adhesive that is not harmful to living tissue.

The term “ear disorder”, as used herein, refers to any disorder that causes hearing or balance loss such as, but not limited to, Meniere's disease, vertigo disorder, ear infections and more.

The term “artificial perilymph”, as used herein, refers to any fluid that simulates the composition and pH of the perilymph.

The term “intratympanic”, as used herein refers to delivery to the tympanic cavity, also known as the middle car, i.e., the small cavity surrounding the small bones that transmit vibrations used in the detection of sound. The tympanic cavity is bounded by the medial wall, which faces the inner car and includes the oval window and the round window, and the lateral wall, which faces the outer car and is formed by the tympanic membrane. Intratympanic delivery or administration is typically by injection through the tympanic membrane.

As used herein, the term “membrane” refers to a Round Window Membrane (RWM), an Oval Window Membrane (OWM), or a combination of both.

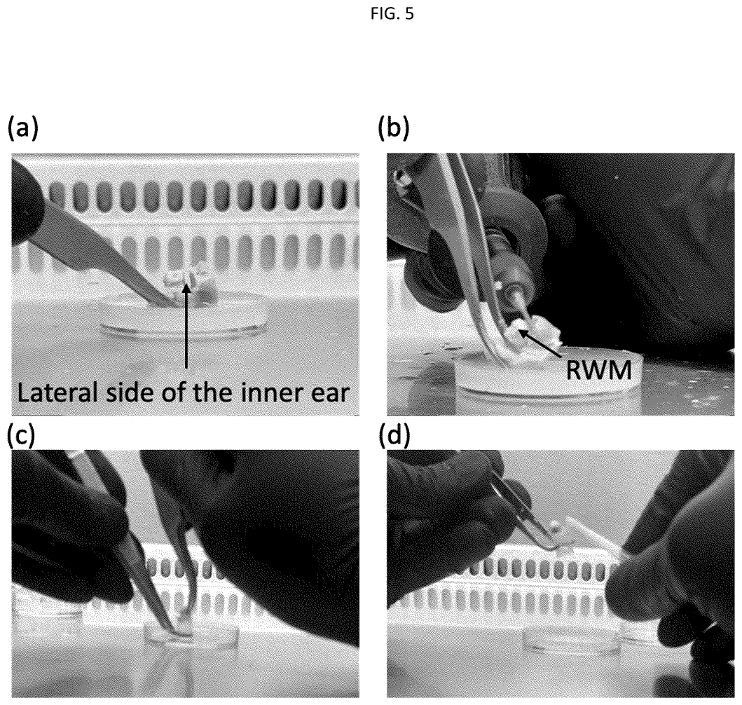

In some embodiments, as shown in FIG. 7, the device includes a first compartment (101), and a second compartment (102), where the first and second compartments are fluidly connected by an aperture. In certain embodiments, such as those represented by the device shown in FIG. 7, the device includes a membrane over the aperture and separating the first and second compartments (103).

In some embodiments of the device disclosed herein, such as that depicted in FIG. 7, the membrane is a mammalian membrane, particularly a membrane from a large mammal, e.g., a dog, cat, sheep, goat, pig, horse, or cow.

In some embodiments of the device disclosed herein, such as that depicted in FIG. 7, the membrane is a porcine (pig) membrane.

The term “mammalian oval window membrane (mOWM)”, as used herein, refers to a membrane or ligament-covered opening from the middle car into the inner car of a mammal.

The term “mammalian round window membrane (mRWM)”, as used herein, refers to an epithelial barrier in the car of a mammal that consists of three layers. The round window is situated below (inferior to) and a little behind (posterior to) the oval window, from which it is separated by a rounded elevation, the promontory.

The term “pRWM” refers to a porcine Round Window Membrane.

The term “POWM” refers to a porcine Oval Window Membrane.

In certain embodiments, the membrane is a mRWM, mOWM, or a combination thereof.

In certain embodiments, the membrane is a pRWM, POWM, or a combination thereof.

In embodiments, where the membrane is a combination of a mRWM and a mOWM, or a combination of pRWM and a pOWM, the membranes can be in contact with each other or can be separated from each other by a distance of up to about 10 mm. Thus, where a combination of membranes is employed, the membranes can be separated by, for example, about 0.1 μm, 0.5 μm, 1 μm, 2 μm, 3 μm, 4 μm, 5 μm, 6 μm, 7 μm, 8, μm, 9 μm, 10, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm or 100 μm, or 1 mm, 2 mm, 3 mm, 4 mm, 5 mm, 6 mm, 7 mm, 8 mm, 9 mm or 10 mm.

In further embodiments, the first and second compartments comprise a Thermoset, thermoplastic polymer, polycarbonate, Poly (methyl methacrylate) (PMMA), Polytetrafluoroethylene (PTFE), polypropylene, metal, or any suitable material.

In exemplary embodiments, a mammalian round window membrane (mRWM) is placed over the aperture between the first and second compartments. The mRWM isolates the first compartment from the second compartment.

In some embodiments, the mRWM is from about 25 μm to about 120 μm thick. In some embodiments, the mRWM is from about 40 μm to about 80 μm thick. In some embodiments, the mRWM is from about 50 μm to about 100 μm thick. In some embodiments, the mRWM is from about 60 μm to about 90 μm thick. In some embodiments, the mRWM is from about 60 μm to about 80 μm thick. In other embodiments, the mRWM is from about 25 μm to about 60 μm thick. In other embodiments, the mRWM is from about 25 μm to about 50 μm thick. In other embodiments, the mRWM is from about 25 μm to about 40 μm thick. In other embodiments, the mRWM is from about 25 μm to about 35 μm thick. In other embodiments, the mRWM is at least about 30 μm thick. In other embodiments, the mRWM is at least about 30 μm thick. In other embodiments, the mRWM is about 30 μm, 35 μm, 40 μm, 45 μm, 50 μm, 55 μm, 60 μm, 65 μm, 70 μm, 75 μm, or 80 μm thick.

In certain embodiments, the mRWM is a porcine round window membrane (pRWM).

In some embodiments, the pRWM is from about 25 μm to about 120 μm thick. In some embodiments, the pRWM is from about 40 μm to about 80 μm thick. In some embodiments, the pRWM is from about 50 μm to about 100 μm thick. In some embodiments, the pRWM is from about 60 μm to about 90 μm thick. In some embodiments, the pRWM is from about 60 μm to about 80 μm thick. In other embodiments, the pRWM is from about 25 μm to about 60 μm thick. In other embodiments, the pRWM is from about 25 μm to about 50 μm thick. In other embodiments, the pRWM is from about 25 μm to about 40 μm thick. In other embodiments, the pRWM is from about 25 μm to about 35 μm thick. In other embodiments, the pRWM is at least about 30 μm thick. In other embodiments, the pRWM is at least about 30 μm thick. In other embodiments, the pRWM is about 30 μm, 35 μm, 40 μm, 45 μm, 50 μm, 55 μm, 60 μm, 65 μm, 70 μm, 75 μm, or 80 μm thick.

The thickness of the RWM and OWM can vary by location on the membrane. Thus, for example, a membrane may have a thickness of 65 μm at one location and a thickness of 75 μm at another.

In certain embodiments, the membrane is a membrane, particularly a round window membrane, that has been removed from a mammal, particularly a pig, that is no more than 20 days old.

In certain embodiments, the membrane, particularly a round window membrane, can be used in the devices disclosed herein for a period of up to about 5 days after it has been excised from the mammal, particularly a pig.

In some embodiments, the membrane, either a mRWM, mOWM or a combination thereof, is supported by a solid support. In particular embodiments, the support is inner ear bone or a biocompatible material. In further embodiments, the excised inner car bone is from about 1-10 mm in diameter. In certain embodiments, the diameter of the support, e.g., bone, is about 1 mm, 2 mm, 3 mm, 4 mm, 5 mm, 6 mm, 7 mm, 8 mm, 9 mm or 10 mm.

In further embodiments, the biocompatible material is any mechanical clamp structure.

In some embodiments, the membrane, which is a mRWM, mOWM or a combination thereof and, when present, supporting material (e.g., inner car bone) are secured over the aperture by an adhesive in an amount suitable to secure the membrane over the aperture. Thus, the membrane may be directly adhered over the aperture using an adhesive, or the membrane may be attached to a support, e.g., bone, and the support is adhered over the aperture. In configurations where the membrane is attached to a support and the support is adhered over the aperture, the membrane can be considered to be indirectly secured over the aperture.

In some embodiments, the membrane, which is a mRWM, mOWM or a combination thereof, is secured over the aperture without an adhesive. In some embodiments, the membrane, which is a mRWM, mOWM or a combination thereof, is secured directly over the aperture by an adhesive in an amount suitable to secure the membrane over the aperture.

In further embodiments, the adhesive used to secure the membrane over the aperture is a biocompatible sealant. Suitable biocompatible sealants for use as the adhesive include zinc phosphate, zinc polycarboxylate, glass ionomer, resin modified glass ionomer, zinc oxide eugenol, a resin cement, or any sealant that tolerates a wet environment and is, preferably, biocompatible.

In some embodiments, the first compartment comprises a solution.

In some embodiments, the second compartment comprises a solution.

In additional embodiments, the solution is different in the first and second compartments.

In certain embodiments, the solution in each compartment is a cell culture media, where the solution in each is the same or different than the solution in the other. The cell culture media is media that provides a favorable artificial environment for maintaining the histotypic architecture and biochemical properties of the cell. Suitable cell culture media comprise serum, supplements, growth factors, anti-mycotic agents, and anti-bacterial agents.

A test compound or test composition may be applied to the test device by providing it to the first compartment in a solution or separately. Where solution and the test compound or test composition are added separately, the solution may be added to the compartment before or after the test compound or composition. Thus, the solution may be free from the test compound or composition or, in other aspects, may contain the test compound or composition. For example, where the device is provided as a kit containing a solution, the kit will typically contain a solution to which a test compound or test composition is added separately and later.

The devices disclosed herein can be considered to be chambers divided into two (2) compartments, each of which may contain fluid, by the membrane. Test compositions, which may be solutions or mixtures, can be added to one side of the membrane (i.e., to a first compartment) and the amounts of various components of the solutions or mixtures, either active compounds or carrier materials, that pass through the membrane into the other (second) compartment can be measured. As described elsewhere herein, these methods can involve measuring with or without removing samples from the compartments.

In some embodiments, a test compound or composition is added to the first compartment or applied to the membrane, which is a mRWM, mOWM or a combination thereof, to identify a suitable therapeutic compound or therapeutic composition or formulation (comprising for example, a drug and a pharmaceutical carrier) for treating a disease or genetic disorder. In this context, this disclosure provides methods for identifying a compound(s) or composition suitable for use as a therapeutic for administration via the car (intratympanic administration) to treat an car disorder or disease, or a genetic disorder in a mammalian subject. These methods comprise applying a test compound or composition to the test device described herein and measuring the amount of the compound or composition that passes through the membrane, which is a mRWM, mOWM or a combination thereof. The devices and methods disclosed herein can be used to test, modify, and develop therapeutic compounds and compositions for delivery to the inner ear via intratympanic administration. The devices and methods can also be used to identify modifications to compounds and compositions to improve delivery of therapeutic compounds. The devices and methods described herein, due to their capability for efficient and rapid use, are an improvement over existing methods for testing and developing compounds and compositions for intratympanic delivery, many of which involve in vivo analyses which are difficult to reproduce.

In some embodiments, the test compound is a nano carrier, a peptide, a mutated virus, mRNA, protein, or small molecule.

In some embodiments, the passage of test compounds through the mRWM and/or mOWM is measured invasively or non-invasively. Non-invasive measurement methods allow for in situ measurements. Invasive measurements typically require fluids to be measured ex situ. Examples of non-invasive measurement methods are optical methods including, for example, fluorescence or Raman spectroscopy. Examples of invasive measurement methods are high sensitivity techniques, including for example, mass spectrometry or genetic analysis. In exemplary embodiments, the disease or genetic disorder is a disease or genetic disorder affecting the ear of a subject.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Methods for Device Fabrication and Characterization

### Animals and Dissection

Yorkshire wild-type pigs (mixed gender) postnatal 0 days (P0), postnatal 20 days (P20), and postnatal 40 days (P40) were dissected. The inner ear was dissected out from the pigs as described in Moatti, A. et al., (Biomed Opt Express 11, 6181-6196 (2020)). First, the skin was removed and a large window on top of the porcine skull was created using a bone striker. The brain was discarded to observe the inner ear. Then, the bone surrounding the inner ear was cut via the striker and excised the porcine inner ear using a bone cutter. Additionally, using a dental drill and 1 mm drill bit described in Moatti, A. et al., (iScience 26, 106789 (2023)), the RWM, including a small portion of the bone around, was excised. The whole drilling procedure was done inside a fresh PBS solution.

### Device Fabrication

After the excision, the surrounding bone around the RWM was dried out using sterile gauze pads and glued into the bottom of a cut 0.5 mm Eppendorf tube using dental cement. The glued RWM was then mounted on top of a Transwell without the mesh and immersed in cell culture media overnight. The media comprised DMEM, 1% antimycotic, and 1% N2. 150 μl of the culture was added on top of the RWM and 1.5 ml on the bottom. The media was changed the next day and the device was ready for experimental work.

### Alamar Blue Viability Test

Viability of the RWM was tested. To perform the viability test, first, the mounted explants were washed with PBS three times. Then 1 ml of fresh media containing 10% alamarBlue was added to the top of the device. The explants were placed in the incubator for 4-6 hr. Absorbance was measured using a plate reader at two wavelengths (570 nm and 600 nm) and percent reduction calculated.

### Permeability Measurements

To measure the permeability of substances, 150 μl culture media was mixed with the substance and placed in the top compartment. This amount of liquid should meet the conditions of continuous infinite dosing (Kelso et al. (Otol. Neurotol. 36(4):694-700 (2015)). The solution was redosed (˜10 μl) after each sampling event. The bottom compartment was filled with 1.5 ml of cell culture media. To monitor the progression of passage, the media in the bottom compartment was sampled. In each sampling event ˜100 μl of liquid was withdrawn from the bottom compartment. Before sampling, the media was pipetted up and down to mix and homogenize. After sampling the media, a 100 μl media was added to the bottom compartment, this dilution in the media was compensated for when calculating the concentrations.

The permeability coefficient (Kp) in m/s was calculated using the following formulae (Kelso et al. 2015):

$${K}_{p} = \frac{Q}{A \cdot t \cdot ( {C}_{0} - {C}_{i} )}$$

where Q is the mass of substance passed through the RWM in time (t) across the exposed area of the RWM (A). The C₀-Ci are the concentrations on the top and bottom of the RWM that can be simplified to C₀ in infinite dosing conditions that here is satisfied by redosing the drug consistently throughout the measurement. Infinite dosing conditions are created by continuously adding drug to the first chamber to maintain substantially the same concentration throughout the experiment.

### Dexamethasone ELISA

A ready-to-use ELISA kit (MyBioSource, MBS2548580) was used to measure Dex concentration in the media collected from the bottom compartment. To generate a standard curve, values of absorbance, which were acquired using a microplate reader (VersaMax), were correlated with known Dex concentrations (0.1-10 ppb). The concentration of Dex in liquid samples were then calculated by comparing the OD of the samples to the standard curve.

### Immunostaining

The RWM tissue explants were fixed using 4% paraformaldehyde (PFA) and then permeabilized and blocked using immunohistochemical blocking buffer with 0.4% Triton for 1 hour at room temperature (RT). Explants were then stained against vimentin and EpCAM at a 1:100 dilution. Explants were incubated with primary antibodies overnight at 4° C. Then, the explants were washed with phosphate buffered saline (PBS) and incubated with fluorophore-labelled secondary antibodies at a 1:200 dilution for 1.5 hours at RT. For nuclear staining, two drops of NucBlue were added to each well after 30 minutes of secondary incubation. The cells were washed with PBS and mounted with ProLong Gold at RT for 4 hours before observation. Explants were imaged using an Olympus FLUOVIEW confocal microscope to detect nuclei, vimentin, and EpCAM. To quantify the RWM thickness, we used IMARIS software.

### Transmission Electron Microscopy (TEM)

For transmission electron microscopy, the tissues were fixed using 4% paraformaldehyde (PFA) and 1% glutaraldehyde in 0.1% sodium cacodylate at 4° C. overnight. The tissues were washed 3× in PBS. Then, the tissues were dehydrated in 50%, 70%, and 95% ethanol (ETOH) for 20-30 min at each step and 2× at 100% ETOH for 30 min. The infiltration (one should employ a specimen rotator) was started by adding the embedding media (Spurr) to the dehydrating fluid (ETOH) left in the vial with tissues (1:2). The mixture was swirled and allowed to stand for 2-3 hr. Then the dehydrating agent/embedding medium was replaced with a 1:1 ratio. The mixture was swirled and allowed to sit overnight. The dehydrating agent/embedding medium was replaced with a 1:3 ratio, swirled, and allowed to sit for 2-3 hr. The mixture was replaced with 100% embedding media for 5-6 hr. or overnight if tissue included bones. The tissues were then polymerized at 70° C. for 24 hr. and cut. A Bio-TEM model HT7800-120 kV transmission electron microscope was used.

### Ex-Vivo RWM Device

The device shown in FIG. 7a-b shows a RWM device comprising a first compartment (101) and a second compartment (102), wherein the first compartment and the second compartment are separated by a membrane (103), and wherein the membrane is attached to a bony structure of the inner ear (201). The first compartment has an upper end with an opening (104), a lower end near the membrane, and walls surrounding the compartment. The second compartment has an upper end that is near the membrane, a lower end with a reservoir, and walls surrounding the compartment. The first compartment and second compartment can be formed as a single unit, as shown in FIG. 7a or as individual parts that can be connected. The walls of the compartments are opaque, clear, or translucent. Each compartment can be colored or colorless, and suitable for optical probing of the liquid.

### Example 2: Characterization of the Porcine Round Window Membrane (pRWM)

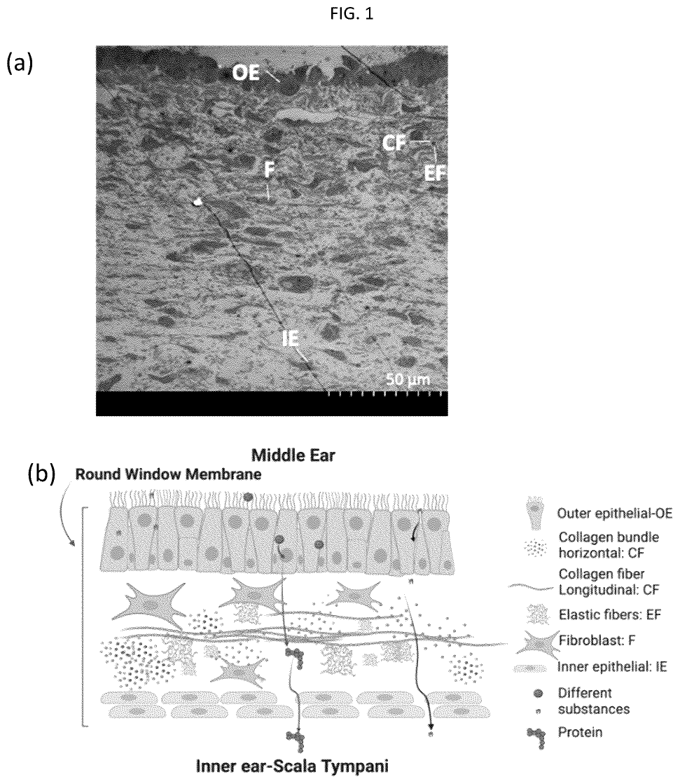

The porcine RWM was characterized using Transmission Electron Microscopy (TEM; FIG. 1a-h) and immunohistochemistry (IHC; FIG. 1i). The RWM structure revealed that RWM consists of three layers with two endodermic epithelium and a middle mesodermal connective tissue layer (FIG. 1a). This three-layer structure is depicted in FIG. 1b. The outer epithelial (OE) cuboidal cells that sit in the vicinity of the middle car cavity form tight junctions that restrict the passage of molecules from the middle to the inner ear (FIG. 1c-e). These cells are active and have mitochondria and rough and smooth endoplasmic reticulum. As shows in FIGS. 1d-f, OE cells with short microvilli likely facilitate absorption and cilia with a 9+2 arrangement of microtubules. The middle layer consists of fibroblastic (Fi) cells and collagen fibers (CF) (FIG. 1g). The node of Ranvier in the middle layer facilitates the conduction of rapid nerve impulses. The inner layer includes the squamous inner epithelial (IE) cells with loose junctions (FIG. 1h). Immunohistochemistry (IHC) was used to verify the presence of epithelial cells and fibroblasts in the porcine RWM (FIG. 1i). As it is shown in higher resolution images, the antibody unveiled the cuboidal shapes of the epithelial cells and elongated shapes of the fibroblasts. The presence of wrinkles is due to the detachment of the membrane from the bony structure during the immunofluorescence staining.

The thickness of the porcine RWM was measured using confocal microscopy cross-sectional images (stained with DAPI). The measured average thickness of the RWM was 71±30 (mean±SD). These results show that the porcine RWM mimics the human RWM anatomy and structure, and is therefore suitable to use as an ex-vivo RWM model.

### Example 3: The Ex-Vivo RWM Device Design, Viability, and Function

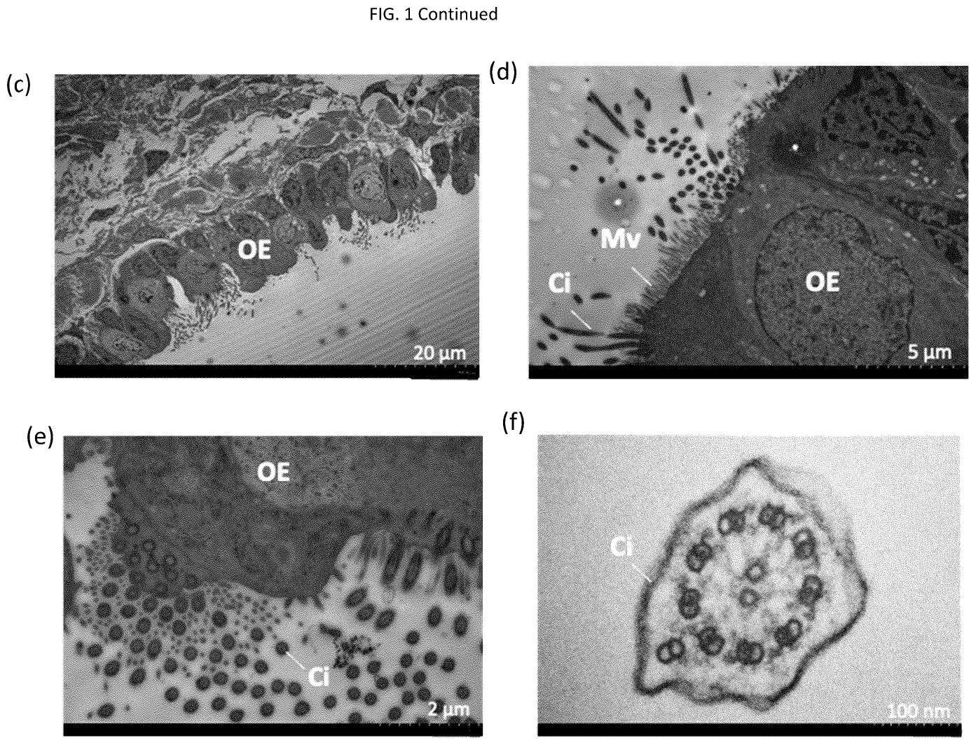

FIGS. 2a-c show the procedure for fabricating a porcine ex-vivo RWM device. As described in Example 1, the porcine inner ear out of the pig skull was dissected and then, using a dental drill, the RWM, including the bony structure that supports it, was excised, and mounted to a transwell insert using dental cement. When the insert is put into a device as disclosed herein, the passage of substances can be tested (FIG. 2c). FIG. 2d displays photographs of postnatal day 0 (P0) porcine inner car and its RWM inside the device where a solution can be passed from the opening of the first compartment through the RWM and collected in the second compartment.

Next, the viability of the excised RWM membranes over time was tested. The viability of the RWM is critical as dead cells could detach and artificially increase the passage of substances. Alamar blue metabolic activity assay was utilized to infer the cellular activity of the RWM explants and cell viability. The results are shown in FIG. 2e, the percent reduction of the Alamar blue for 7 explants up to 5 days were calculated. Cell metabolic activity in the explanted membranes under the conditions described here decreases with time, and by day 5 it is only ˜20% of the initial activity. Thus, passage of substances was evaluated through the membrane in the device for no more than 3 days.

To establish the functionality of the device, the passage of a known high permeability therapeutic, dexamethasone (Dex) was tested. Dexamethasone has been shown to pass through the RWM in multiple animal models and humans (Nordang, et al., Otology & Neurotology: Official Publication of the American Otological Society, American Neurotology Society [and] European Academy of Otology and Neurotology 24, 339-343 (2003); Salt, A. N. et al., Audiol Neurootol 23, 245-257 (2018); Sun, J. et al., Chinese Medical Journal 120, 2284-2289 (2007); Tang, B. et al., Am J Otolaryngol 39, 676-678 (2018). As expected, Dex passes quickly <0.5 h through the porcine RWM.

The permeability coefficient (Kp) calculated as described in Example 1, of RWM to Dex at different time points and plotted is shown in FIG. 2g. The average of the Kp at two different regimes of 0.5-2.5 hr. and after 20 hr. is 7.0±4.3 c-7 m/s and 1.0±0.5 e-7 m/s (mean±SD), respectively.

### Example 4: Measuring the Passage of a Low Permeability Substance Through the Ex-Vivo Round Window Membrane

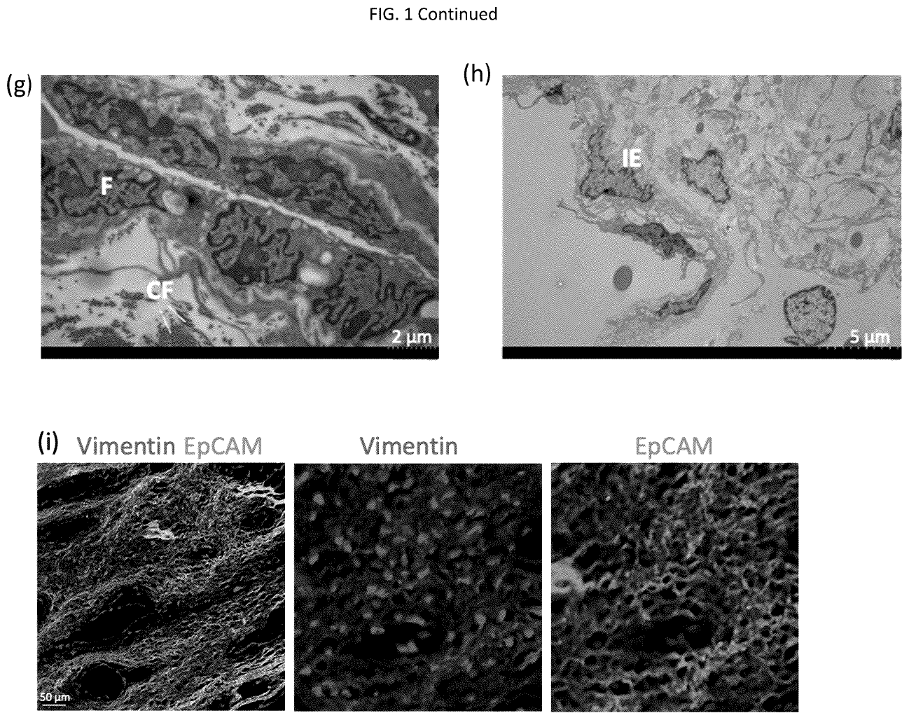

The passage of a larger molecule with lower permeability through the RWM explants—dexamethasone fluorescein (DexF) was also tested. The difference between the molecular structures of Dex (392 g/mol) and DexF (841 g/mol) is shown schematically (FIG. 3a). Based on experiments in guinea pigs, DexF is expected to exhibit a lower passage through the RWM compared to Dex (Li et al. 2018) (FIG. 3a). To test DexF passage, 0.5 mM DexF solution was added to the top compartment of each of eight devices, where the devices included RMW from pigs of different ages (P0, P20, and P40). The media at the bottom of the compartment was subsequently collected and analyzed using a plate reader at multiple time points (FIG. 3b). The DexF passed through the membrane less efficiently than Dex. The Kp for all the explants was calculated at different time points as shown in FIG. 3c. The average Kp values up to 4.5 hr. from the introduction of DexF, and above were calculated to be 3.7±3.7 e-9 m/s and 4.2±2.4 e-10 m/s (mean±SD), respectively. The reported Kp for DexF is found to be two orders of magnitude lower than for Dex. Similar to Dex, the permeability decreases over time by one order of magnitude (FIG. 3c). A significant decrease in the Kp of the P40 was observed in comparison to P0-20 explants using a one-way ANOVA test. The passage of fluorescein alone is similar to DexF. The viability of RWM explants has been shown to be paramount to in vivo passage of substances through the RWM in guinea pig models (Zhang et al. Int J Nanomedicine. 2018; 13:479-492). Passage through the membrane includes a series of active uptake events including both endocytic and exocytic processes that affect the permeability of the test compound.

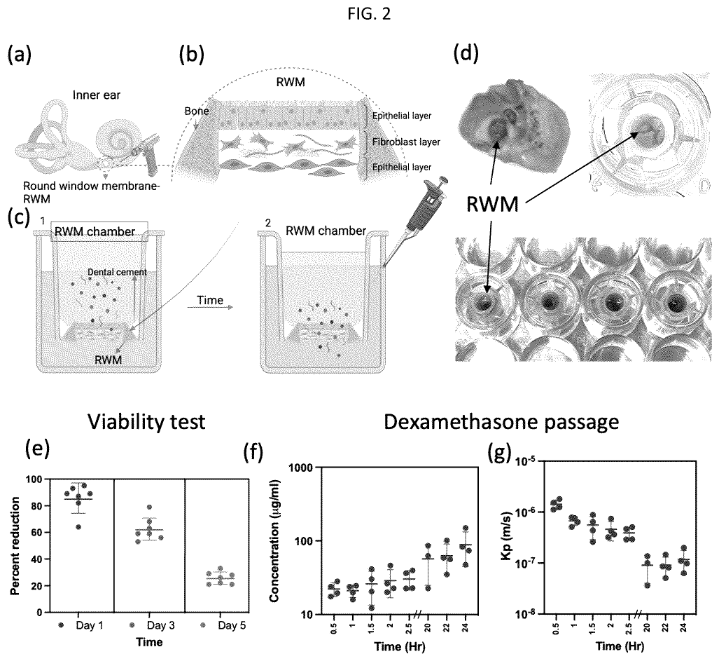

Round window membrane viability was tested to determine if it affected DexF passage. The passage of DexF was tested on two healthy yet leaky explants that do not hold the solution on top properly and on two explants with a lowered metabolic activity (percent reduction, PR—of ˜20%) as shown schematically in FIG. 4a. Passage of DexF through membranes having healthy but lowered metabolic activity and leaky membranes is shown in FIG. 4b. Passage of DexF through the lowered metabolic activity and leaky membranes was higher than healthy and non-leaky explants. The Kp values (FIG. 4c) and they were calculated for two-time points (up to 4.5 hr. from the introduction of DexF, and above) 6.6±7.0 e-8 m/s and 1.3±0.8 e-8 m/s for lowered metabolic activity explants and 1.7±0.8 e-7 m/s and 5.0±0.1 e-8 m/s for leaky explants (mean±SD). In comparison with the membranes, both the leaky and the lower metabolic activity groups have Kp values that are two orders of magnitude higher. This result shows that high viability of the RWM is crucial for assessing the permeability of materials, and these passage values provided suitable exclusion criteria. The membranes used in the RWM/OWM devices described herein are viable for use up to about 5 days post excision.

### Example 5: Measuring the Passage of Low Permeability Substance Through the Round Window Membrane In-Vivo

To demonstrate equivalence of the membrane test device disclosed herein (ex vivo) to an in vivo porcine Round Window Membrane, the results of ex vivo permeability measurements made with the disclosed device are compared with data obtained from a porcine car in vivo.

Dexamethasone was injected intratympanically to a porcine inner ear in vivo. About 20 μl of perilymph was collected using microcapillary tubes (5 μl volume each) 0.5-hour post injection. The concentration of dexamethasone was measured in the perilymph using Mass spectrometry.

Separately, dexamethasone (100 μg/ml) was applied to the upper chamber of a RWM test device and a sample of liquid was collected from the lower chamber after 0.5 hour and the concentration of dexamethasone in the lower chamber was determined.

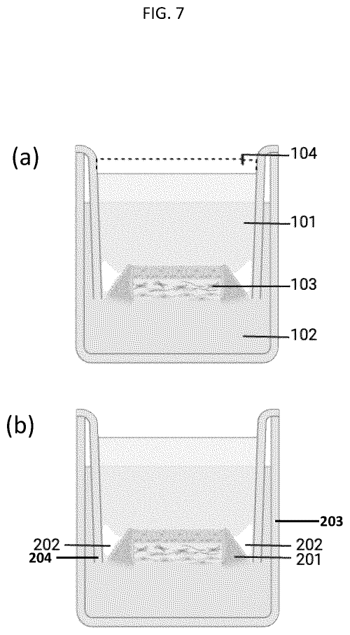

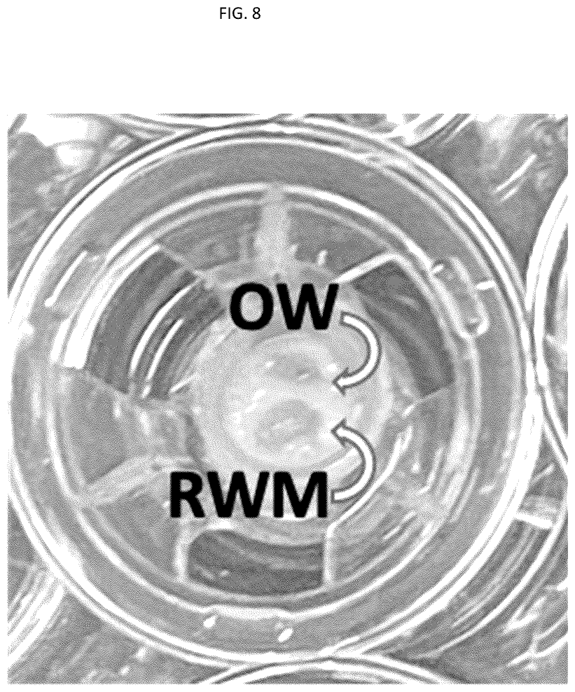

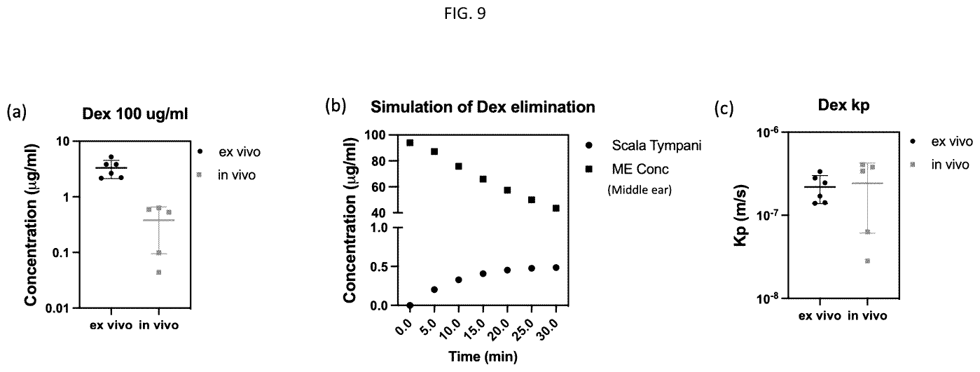

The concentration of dexamethasone in the lower chamber is determined using mass spectrometry and compared in FIG. 9a to the in vivo concentration of dexamethasone inside the inner ear. The difference in concentration between the ex vivo and in vivo measurements can be explained by the fact that the ex-vivo chamber does not account for the fluid removal processes of the middle car, thus higher concentration is expected in the ex-vivo chamber. To account for fluid removal in the middle car this process was simulated.

A simulation of in vivo dexamethasone removal from the middle car was carried out by FluidSim4. The results of the in vivo simulation after 0.5 hour are shown graphically in FIG. 9b.

Using the simulated data generated as described above and shown in FIG. 9b, in vivo dexamethasone permeability was calculated. The introduced drug, C₀, in the calculation of permeability, kp, is adjusted based on the amount that is removed from the middle car. In vivo permeability is shown comparatively in FIG. 9c with ex vivo permeability determined using the RWM test device of this disclosure. As can be seen, the ex-vivo and in-vivo measurements were comparable, confirming the predictability of the ex-vivo chamber.

Bird et al. (Otol Neurotol. 2011 August;32(6):933-6), reports that, after intratympanic dexamethasone sodium phosphate (DSP, 0.4-1.8 ml of a 4 mg/ml solution) injection in humans, the dexamethasone concentration in perilymph is about 1.4 μg/and the DSP concentration in perilymph (20 μl) is about 7.5 μg/mL. The dexamethasone concentration in the porcine inner ear perilymph (20 μl) after in-vivo intratympanic injection of DSP (1 ml of 4 mg/ml) was about 5.06 μg/mL (n=3; range, 0.52-13.10) and the concentration of DSP in the perilymph was about 1.21 μg/mL (n=3; range, 0.25-2.60). The porcine and human mean total concentrations of the dexamethasone and DSP in the perilymph are very similar. These results demonstrate the validity of using the disclosed membrane test device for modeling drug passage across the human RWM and/or OWM.

### Itemized List of Embodiments

A first embodiment is a test device comprising: A first compartment and a second compartment, wherein the first compartment is fluidly connected to the second compartment by an aperture between the first compartment and the second compartment; and a membrane selected from a mammalian round window membrane (mRWM), a mammalian oval window membrane (mOWM), or a combination of a mRWM and a mOWM; wherein the membrane is provided over the aperture such that the membrane isolates the first compartment from the second compartment.

A second embodiment is the test device according to the first embodiment, wherein the membrane is a large mammal round window membrane, a large mammal oval window membrane, or a combination of a large mammal round window membrane and a large mammal oval window membrane.

A third embodiment is the test device according to the first embodiment, wherein the membrane is a porcine round window membrane (pRWM), a porcine oval window membrane (pOWM), or a combination of a pRWM and a pOWM.

A fourth embodiment is a test device according to the first embodiment, wherein the round window membrane has a thickness of at least about 65 μm.

A fifth embodiment is the test device of any one of the first through fourth embodiment, wherein the membrane is a round window membrane having a thickness of at least 30 μm.

A sixth embodiment is the test device of any one of the first through fourth embodiment, wherein the membrane is an oval window membrane having a thickness of at least 30 μm.

A seventh embodiment is the test device any one of the first through fourth embodiment, wherein the membrane is a combination of a round window membrane and an oval window membrane.

An eight embodiment is the test device according to any one of the preceding embodiments, wherein the device further comprises a support for the membrane.

A ninth embodiment is the test device according to any one of the preceding embodiments, wherein the support is inner ear bone and/or any biocompatible material.

A tenth embodiment is the test device according to any one of the preceding embodiments, wherein the membrane is secured over the aperture by an adhesive.

An eleventh embodiment is the test device according to the tenth embodiment, wherein the adhesive is any biocompatible sealant.

A twelfth embodiment is the test device according to the eleventh embodiment, wherein the biocompatible sealant is a zinc phosphate, zinc polycarboxylate, glass ionomer, resin modified glass ionomer, zinc oxide eugenol, or a resin cement.

A thirteenth embodiment is the test device according to the first embodiment, wherein
the first compartment further comprises a first solution; and
the second compartment further comprises a solution,
wherein the solution in the second compartment is the same or different than the solution in the first compartment.

A fourteenth embodiment is the test device according to any one of the preceding embodiments, further comprising apparatus for measuring the concentration of a test compound in the first compartment, the second compartment, or both the first and second compartments.

A fifteenth embodiment is the test device according to the thirteen or fourteenth embodiments, wherein the first solution is a cell culture media comprising serum, supplements, growth factors, anti-mycotic agents, and anti-bacterial agents.

A sixteenth embodiment is the test device according to the fifteenth embodiment, wherein the first solution contains a test compound or test composition.

A seventeenth embodiment is the test device according the fifteenth embodiment, wherein the first solution is free of a test compound or test composition.

An eighteen embodiment is a method of identifying a compound or composition suitable for use as a therapeutic via intratympanic administration to treat an ear disorder or disease, or a genetic disorder in a mammalian subject, the method comprising applying a test compound or test composition comprising a test compound to the test device of any one of the preceding embodiments and measuring the amount of test compound that passes through the membrane.

A nineteenth embodiment is the method according to the eighteenth embodiment, wherein the measuring comprises detecting test compound in solution in the second compartment.

A twentieth embodiment in the method according to the eighteenth embodiment, wherein the measuring comprises determining the concentration of the test compound in solution in the second compartment.

A twenty-first embodiment is the method according to the eighteenth embodiment, further comprising calculating the permeability of the test compound through the membrane.

A twenty-second embodiment is the method of identifying a compound or composition suitable for use as a therapeutic via intratympanic administration to treat an ear disorder or disease, or a genetic disorder in a mammalian subject, the method comprising applying a test compound or composition comprising a test compound to the test device of any the first through seventeenth embodiments and determining permeability of test compound that passes through the membrane.

A twenty-third embodiment is the method according to the eighteenth or twenty-second embodiment, wherein the measuring comprises determining active passage of the test compound through the membrane.

A twenty-fourth embodiment is the method of treating an inner ear disease or disorder in a subject, the method comprising administering to a subject in need thereof a therapeutically effective amount of a compound or composition identified by the method of the eighteenth embodiment.

A twenty-fifth embodiment is a method of assembling a test device, the method comprising:
securing membrane selected from a mammalian round window membrane (mRWM), a mammalian oval window membrane (mOWM), mRWM, mOWM, and a combination of a mRWM and a mOWM, over an opening of a first compartment, wherein the first compartment is fluidly connected to a second compartment.

A twenty-sixth embodiment is the method according to the twenty-fifth embodiment, wherein the membrane is a large mammal round window membrane, a large mammal oval window membrane, or a combination of a large mammal round window membrane and a large mammal oval window membrane.

A twenty-seventh embodiment is the method according to the twenty-fifth embodiment, wherein the membrane is a porcine membrane.

A twenty-eighth embodiment is the method according to the twenty-fifth embodiment, wherein the membrane is a porcine round window membrane (pRWM).

A twenty-ninth embodiment is the method according to the twenty-fifth embodiment, where in the membrane is a porcine oval window membrane (pOWM).

A thirtieth embodiment is the method according to the twenty-fifth embodiment, where in the membrane is a combination of a pRWM and a pOWM.

A thirty-first embodiment is the method according to any of the twenty-fifth through thirtieth embodiments, further comprising mounting the membrane to the first compartment with an adhesive.

A thirty-second embodiment is the method according to the thirty-first embodiment, wherein the membrane is attached to a support.

A thirty-third embodiment is the method according to thirty-second embodiment, wherein the support is mounted to the first compartment with an adhesive.

A thirty-fourth embodiment is the method according to the twenty-fifth embodiment, further comprising adding cell culture media, or a synthetic perilymph, to the first compartment, the second compartment, or both compartments.

A thirty-fifth embodiment is the method of testing delivery of a therapeutic compound or therapeutic composition into the inner car, the method comprising applying the therapeutic composition or therapeutic compound or test composition to the test device of the first embodiment and measuring the therapeutic compound or therapeutic composition that passes through the membrane.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification, and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

The disclosure of any document, e.g., a journal article or patent document, mentioned herein is incorporated herein by reference in its entirety.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth herein.

We claim:

1. A test device comprising:
A first compartment and a second compartment, wherein the first compartment is fluidly connected to the second compartment by an aperture between the first compartment and the second compartment; and
a membrane selected from a mammalian round window membrane (mRWM), a mammalian oval window membrane (mOWM), or a combination of a mRWM and a mOWM;
wherein the membrane is provided over the aperture such that the membrane isolates the first compartment from the second compartment.

2. A test device according to claim 1, wherein the membrane is a large mammal round window membrane, a large mammal oval window membrane, or a combination of a large mammal round window membrane and a large mammal oval window membrane.

3. A test device according to claim 1, wherein the membrane is a porcine round window membrane (pRWM), a porcine oval window membrane (pOWM), or a combination of a pRWM and a pOWM.

4. A test device according to claim 1, wherein the round window membrane has a thickness of at least about 65 μm.

5. A test device according to claim 1, wherein the membrane is a round window membrane having a thickness of at least 30 μm.

6. A test device according to claim 1, wherein the membrane is an oval window membrane having a thickness of at least 30 μm.

7. A test device according to claim 1, wherein the membrane is a combination of a round window membrane and an oval window membrane.

8. A test device according to claim 1, wherein the device further comprises a support for the membrane.

9. A test device according to claim 1, wherein the support is inner ear bone or any biocompatible material.

10. A test device according to claim 1, wherein the membrane is secured over the aperture by an adhesive.

11. A test device according to claim 10, wherein the adhesive is any biocompatible sealant.

12. A test device according to claim 11, wherein the biocompatible sealant is a zinc phosphate, zinc polycarboxylate, glass ionomer, resin modified glass ionomer, zinc oxide eugenol, or a resin cement.

13. The test device according to claim 1, wherein
the first compartment further comprises a first solution; and
the second compartment further comprises a solution,
wherein the solution in the second compartment is the same or different than the solution in the first compartment.

14. A test device according to claim 1, further comprising apparatus for measuring the concentration of a test compound in the first compartment, the second compartment, or both the first and second compartments.

15. A test device according to claim 13, wherein the first solution is a cell culture media comprising serum, supplements, growth factors, anti-mycotic agents, and anti-bacterial agents.

16. A test device according to claim 15, wherein the first solution contains a test compound or test composition.

17. A test device according to claim 15, wherein the first solution is free of a test compound or test composition.

18. A method of identifying a compound or composition suitable for use as a therapeutic via intratympanic administration to treat an ear disorder or disease, or a genetic disorder in a mammalian subject, the method comprising applying a test compound or test composition comprising a test compound to the test device according to claim 1 and measuring the amount of test compound that passes through the membrane.

19. A method according to claim 18, wherein the measuring comprises detecting test compound in solution in the second compartment.

20. A method according to claim 18, wherein the measuring comprises determining the concentration of the test compound in solution in the second compartment.

21. A method according to claim 18, further comprising calculating the permeability of the test compound through the membrane.

22. A method of identifying a compound or composition suitable for use as a therapeutic via intratympanic administration to treat an ear disorder or disease, or a genetic disorder in a mammalian subject, the method comprising applying a test compound or composition comprising a test compound to the test device according to claim 1 and determining permeability of test compound that passes through the membrane.

23. A method according to claim 22, wherein the measuring further comprises determining the extent of active passage of the test compound through the membrane.

24. A method of treating an inner ear disease or disorder in a subject, the method comprising administering to a subject in need thereof a therapeutically effective amount of a compound or composition identified by the method of claim 18.

25. A method of assembling a test device, the method comprising:
securing membrane selected from a mammalian round window membrane (mRWM), a mammalian oval window membrane (mOWM), mRWM, mOWM, and a combination of a mRWM and a mOWM, over an opening of a first compartment, wherein the first compartment is fluidly connected to a second compartment.

26. A method according to claim 25, wherein the membrane is a large mammal round window membrane, a large mammal oval window membrane, or a combination of a large mammal round window membrane and a large mammal oval window membrane.

27. A method according to claim 25, wherein the membrane is a porcine membrane.

28. A method according to claim 25, wherein the membrane is a porcine round window membrane (pRWM).

29. A method according to claim 25, where in the membrane is a porcine oval window membrane (pOWM).

30. A method according to claim 25, where in the membrane is a combination of a pRWM and a pOWM.

31. A method according to claim 25, further comprising mounting the membrane to the first compartment with an adhesive.

32. A method according to claim 31, wherein the membrane is attached to a support.

33. A method according to claim 32, wherein the support is mounted to the first compartment with an adhesive.

34. A method according to claim 25, further comprising adding cell culture media, or a synthetic perilymph, to the first compartment, the second compartment, or both compartments.

35. A method of testing delivery of a therapeutic compound or therapeutic composition into the inner ear, the method comprising applying the therapeutic composition or therapeutic compound or test composition to the test device of claim 1 and measuring the therapeutic compound or therapeutic composition that passes through the membrane.

36. A method of identifying a compound or composition suitable for use as a therapeutic via intratympanic administration to treat an ear disorder or disease, or a genetic disorder in a mammalian subject, the method comprising applying a test compound or test composition comprising a test compound to the test device according to claim 1 and determining the permeability of test compound through the membrane.
